(19) United States
(12) Patent Application Publication
(10) Pub. No.: US 2025/0160788 A1
(43) Pub. Date: May 22, 2025

(54) METHOD AND SYSTEM FOR 3D REGISTERING OF ULTRASOUND PROBE IN LAPAROSCOPIC ULTRASOUND PROCEDURES AND APPLICATIONS THEREOF

(71) Applicant: EDDA TECHNOLOGY, INC., PRINCETON, NJ (US)

(72) Inventors: Guo-Qing Wei, Plainsboro, NJ (US); Xiaonan Zang, Princeton, NJ (US); Li Fan, Belle Mead, NJ (US); Xiaolan Zeng, Princeton, NJ (US); Jianzhong Qian, Princeton Junction, NJ (US)

(21) Appl. No.: 18/517,735

(22) Filed: Nov. 22, 2023

(51) Int. Cl. A61B 8/00; A61B 8/08; A61B 8/12

(52) U.S. Cl. CPC A61B 8/4263; A61B 8/0891; A61B 8/12; A61B 8/463; A61B 8/466

(57) ABSTRACT

System, method, medium, and implementation of registering an ultrasound probe in a laparoscopic ultrasound procedure and application thereof is disclosed. The two-dimensional (2D) location of the ultrasound probe in a 2D laparoscopic (LP) image is detected, where the LP camera is previously calibrated in a three-dimensional (3D) space. The 3D pose of the ultrasound probe as deployed is estimated and registered in the 3D space based on the detected 2D location of the ultrasound probe and an ultrasound model for the ultrasound probe.

### BACKGROUND

### 1. Technical Field

The present teaching generally relates to computers. More specifically, the present teaching relates to signal processing.

### 2. Technical Background

With the advancement of technologies, more and more tasks are now performed with the assistance of computers. Different industries have benefited from such technological advancement, including the medical industry, where large volume of image data, acquired via modern sensing techniques and capturing anatomical information of a patient, may be processed by computers to identify useful information of different types and utilized in different applications such as visualization in a more effective manner to assist in diagnosis, pre-surgical planning, and assistance in surgical instrument navigation during a surgery. With the advancement in signal processing techniques, it is now frequent that computers can automatically identify anatomical structures of interest (e.g., organs, bones, blood vessels, or abnormal nodule) from different sensed data such as images, obtain measurements for each object of interest (e.g., dimension of a nodule growing in an organ), and visualize features of interest (e.g., three-dimensional (3D) visualization of an abnormal nodule) with sufficient detail in any desired angle.

Such information may be used for a wide variety of purposes. For example, 3D models may be constructed for a target organ, representing the physical characteristics of an organ (e.g., a liver in terms of its volume, shape, or size along different dimensions), its parts thereof (e.g., a nodule growing inside a liver, vessel structures inside and near the organ) as well as spatial relationships with other surrounding anatomical structures. Such 3D models may be leveraged in different applications. For example, organ 3D models may be used in pre-surgical planning to derive a surgical path from the skin of a patient and a target such as a nodule in an organ to be surgical removed in a surgery. During a surgery, a 3D model for an organ may be rendered to show a user (e.g., a surgeon) certain desired information when the 3D model is projected in an appropriate perspective. In addition, while laparoscopic sensing techniques made it possible to acquire visual information about organs inside patients to provide guidance to perform non-invasive surgeries, an ultrasound probe now may be deployed in a laparoscopic ultrasound (LUS) surgical setting to help to see what is under the surface of an organ visible to a laparoscopic camera.

A LUS setting is illustrated in FIG. 1A, where a patient 120 lying on a surgical table 110 during a laparoscopic procedure, in which a laparoscopic camera 130 and an ultrasound probe 140 may be inserted into the body of the patient 120 to acquire two-dimensional (2D) images, including laparoscopic images 150 and ultrasound images 160, respectively. The 2D laparoscopic images 150 provide what is visible in front of a surgical instrument 170 and the ultrasound images 160 may provide information sensed by the ultrasound probe 140 beneath the surface of an organ visible to the laparoscopic camera 130. The laparoscopic images 150 and the ultrasound images 160 together provide visual guidance to a surgeon in terms of how the surgeon may manipulate the surgical instrument 170 during the surgery. Due to the proximity of the laparoscopic camera 130 and the ultrasound probe 140, the visual information in the laparoscopic images 150 may also include the views of the ultrasound probe 140.

Ultrasound images capture what is present at a certain depth from an ultrasound probe and are known to be noisy with often only incomplete information on each image. Because of these reasons, relying on ultrasound images to piece together information to recognize anatomical structures such as blood vessels require substantial experience and skill. In addition, even when a skilled surgeon may be able to tell what correspond to blood vessels and what correspond to other anatomical structures, it is very difficult if not impossible to ascertain the type of each vessel. FIG. 1B shows an example ultrasound image corresponding to a slice of an organ where some 2D structures may be identified by human and based on experience, such structures may be manually labeled as, e.g., vessels 180 and 185 and a piece of boundary of a tumor 190. In this example, the labels assigned to vessels 180 and 185 may correspond to portal vein and hepatic vein based on observations and experience of a user. In some surgeries, accurate labeling of the vessels may be crucially important. For instance, in some operations, a surgeon may need to clamp a portal vein prior to remove a tumor in the same vicinity. At present time, labeling different anatomical structures in ultrasound images are still performed manually by humans.

Thus, there is a need for a solution that improves the current state of the art discussed above.

### SUMMARY

The teachings disclosed herein relate to methods, systems, and programming for information management. More particularly, the present teaching relates to methods, systems, and programming related to hash table and storage management using the same.

In one example, a method, implemented on a machine having at least one processor, storage, and a communication platform capable of connecting to a network for registering an ultrasound probe in a laparoscopic ultrasound procedure and application thereof is disclosed. The two-dimensional (2D) location of the ultrasound probe in a 2D laparoscopic (LP) image is detected, where the LP camera is previously calibrated in a three-dimensional (3D) space. The 3D pose of the ultrasound probe as deployed is estimated and registered in the 3D space based on the detected 2D location of the ultrasound probe and an ultrasound model for the ultrasound probe.

In a different example, a system is disclosed for registering an ultrasound probe in a laparoscopic ultrasound procedure and application thereof. The system includes an LP U-probe location detector and a 3D U-probe pose estimator. The LP U-probe location detector is provided for detecting a two-dimensional (2D) location of an ultrasound probe visible in a 2D laparoscopic (LP) image acquired by an LP camera previously calibrated in a three-dimensional (3D) space and inserted into a patient's body during a laparoscopic ultrasound (LPUS) procedure. The 3D U-probe pose estimator is provided for estimating a 3D pose of the ultrasound probe deployed in the LPUS procedure based on the detected 2D location of the ultrasound probe and an ultrasound model for the ultrasound probe.

Other concepts relate to software for implementing the present teaching. A software product, in accordance with this concept, includes at least one machine-readable non-transitory medium and information carried by the medium. The information carried by the medium may be executable program code data, parameters in association with the executable program code, and/or information related to a user, a request, content, or other additional information.

Another example is a machine-readable, non-transitory and tangible medium having information recorded thereon for registering an ultrasound probe in a laparoscopic ultrasound procedure and application thereof. The information, when read by the machine, causes the machine to perform the following steps. The two-dimensional (2D) location of the ultrasound probe in a 2D laparoscopic (LP) image is detected, where the LP camera is previously calibrated in a three-dimensional (3D) space. The 3D pose of the ultrasound probe as deployed is estimated and registered in the 3D space based on the detected 2D location of the ultrasound probe and an ultrasound model for the ultrasound probe.

Additional advantages and novel features will be set forth in part in the description which follows, and in part will become apparent to those skilled in the art upon examination of the following and the accompanying drawings or may be learned by production or operation of the examples. The advantages of the present teachings may be realized and attained by practice or use of various aspects of the methodologies, instrumentalities and combinations set forth in the detailed examples discussed below.

### BRIEF DESCRIPTION OF THE DRAWINGS

The methods, systems and/or programming described herein are further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1A shows an exemplary laparoscopic procedure setting with also an ultrasound probe deployed to acquire information;

FIG. 1B shows an ultrasound image with some 2D structures with human provided labels;

FIG. 2A depicts an exemplary high level system diagram of a framework for estimating a 3D ultrasound probe pose (USPP) based on 2D laparoscopic images and application thereof to vessel labeling, in accordance with an embodiment of the present teaching;

FIG. 2B is a flowchart of an exemplary process for estimating a 3D USPP based on 2D laparoscopic images and application in vessel labeling, in accordance with an embodiment of the present teaching;

FIG. 3A is an exemplary high level system diagram of an LP U-probe location detector, in accordance with an embodiment of the present teaching;

FIG. 3B shows exemplary detected ultrasound probe in a laparoscopic image, in accordance with an embodiment of the present teaching;

FIG. 3C shows an exemplary 2D region representing a detected ultrasound probe with some features therein and a 2D location of the ultrasound probe defined as a centroid of the 2D region, in accordance with an embodiment of the present teaching;

FIG. 3D is a flowchart of an exemplary process of an LP U-probe location detector, in accordance with an embodiment of the present teaching;

FIG. 3E depicts an exemplary scheme of determining a 3D coordinate of a U-probe by back projecting a 2D U-probe location detected from a laparoscopic image, in accordance with an embodiment of the present teaching;

FIGS. 3F-3G illustrate a 3D structure of an opening on an ultrasound probe and properties thereof that change with respect to viewing angles, in accordance with different embodiments of the present teaching;

FIG. 4A depicts an exemplary high level system diagram of a 3D U-probe pose estimator, in accordance with an embodiment of the present teaching;

FIG. 4B is a flowchart of an exemplary process of a 3D U-probe pose estimator, in accordance with an embodiment of the present teaching;

FIG. 5A depicts an exemplary high level system diagram of a 2D vessel label generator, in accordance with an embodiment of the present teaching;

FIGS. 5B-5C show 2D structures detected from a 2D ultrasound image and vessel boundaries thereof, in accordance with an embodiment of the present teaching;

FIG. 5D illustrates an exemplary virtual ultrasound slice image with certain vessel structures with labels generated based on an estimated 3D U-probe pose, in accordance with an embodiment of the present teaching;

FIG. 5E illustrates virtual vessel structured rendered using a 3D model superimposed on vessel structures extracted from an ultrasound image, in accordance with an embodiment of the present teaching;

FIG. 5F is a flowchart of an exemplary process of a 3D U-probe pose estimator, in accordance with an embodiment of the present teaching;

FIG. 5G is a flowchart of an exemplary process of a 2D vessel label generator, in accordance with an embodiment of the present teaching;

FIG. 6 is an illustrative diagram of an exemplary mobile device architecture that may be used to realize a specialized system implementing the present teaching in accordance with various embodiments; and

FIG. 7 is an illustrative diagram of an exemplary computing device architecture that may be used to realize a specialized system implementing the present teaching in accordance with various embodiments.

### DETAILED DESCRIPTION

In the following detailed description, numerous specific details are set forth by way of examples in order to facilitate a thorough understanding of the relevant teachings. However, it should be apparent to those skilled in the art that the present teachings may be practiced without such details. In other instances, well known methods, procedures, components, and/or system have been described at a relatively high-level, without detail, in order to avoid unnecessarily obscuring aspects of the present teachings.

The present teaching discloses exemplary methods, systems, and implementations for estimating the 3D USPP in a LUS procedure and use of the estimated USPP in labeling different anatomical structures observed in ultrasound images in a LUS procedure. A LUS procedure has been used for different surgeries such as laparoscopic liver resection because the use of an ultrasound probe may provide views of the anatomical structures such as vessels and tumors beneath the surface of an organ. Prior to a surgery, 3D organ models may be constructed based on 3D medical image data obtained from scans such as computerized tomography (CT) or magnetic resonance imaging (MRI). Ultrasound and CT registration during a surgery may be possible. However, as the images acquired by a laparoscopic camera in a LUS procedure have limited field of view, it is usually not possible because laparoscopic images do not reveal adequate anatomical structures. On the other hand, ultrasound images acquired via an ultrasound probe in a LUS procedure are generally quite noisy with only partial information so that they also cannot provide adequate information on anatomical structures that a user needs to see during a surgery.

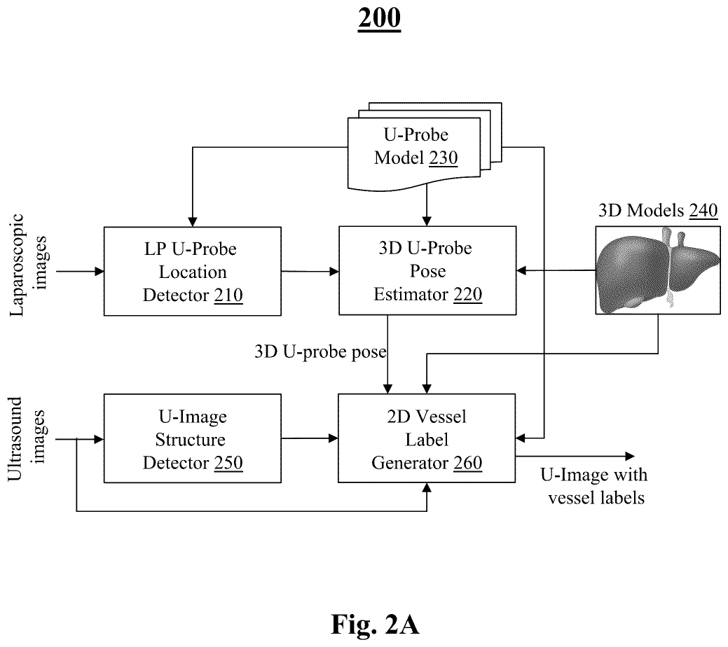

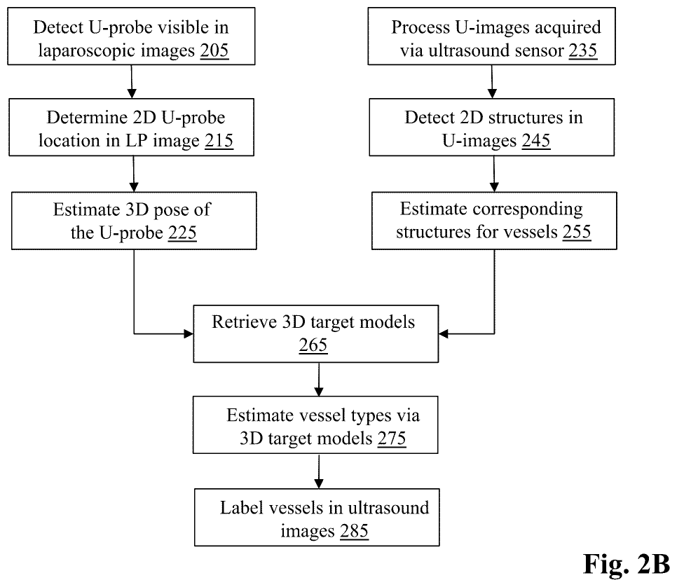

The present teaching discloses a framework for estimating the 3D USPP and then leverage such an estimated 3D USPP to label the anatomical structures partially observed in 2D ultrasound images. The estimation of the 3D USPP is performed based on an ultrasound probe captured in 2D laparoscopic images by a laparoscopic camera in the LUS procedure. When the pose of the laparoscopic camera is estimated with respect to the 3D models of a target organ (the estimation is not the scope of the present teaching), each pixel in a laparoscopic image including a 2D location of the ultrasound probe as detected in the 2D laparoscopic image may be mapped to a 3D location in the 3D space of the 3D models. To determine the orientation of the ultrasound probe, a 3D model for a feature on the ultrasound probe may be used. In some embodiments, such a feature may correspond to an opening located on the ultrasound probe and may be detected from the 2D laparoscopic image, which may be used to compare with different projections of the 3D model of the opening using different orientations from the estimated 3D location of the ultrasound probe. One of the projections created using a particular orientation may be identified to correspond to the best match so that the orientation may be identified as the orientation of the ultrasound probe (i.e., a rotation angle around the US probe axis). With the 3D location and orientation of the ultrasound probe estimated by leveraging its 2D features observed in 2D laparoscopic images, the 3D USPP may be determined. Details related to estimating a 3D USPP of an ultrasound probe are disclosed with reference to FIGS. 2-4B.

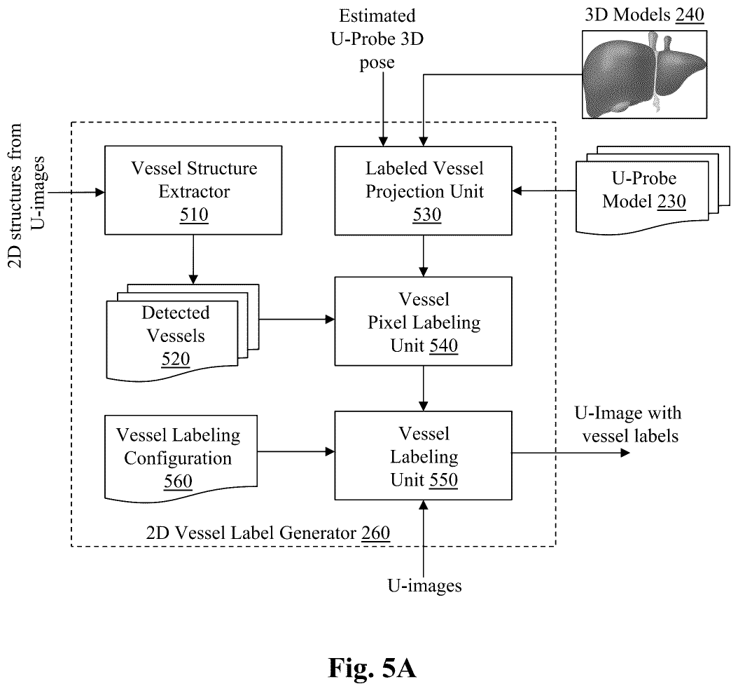

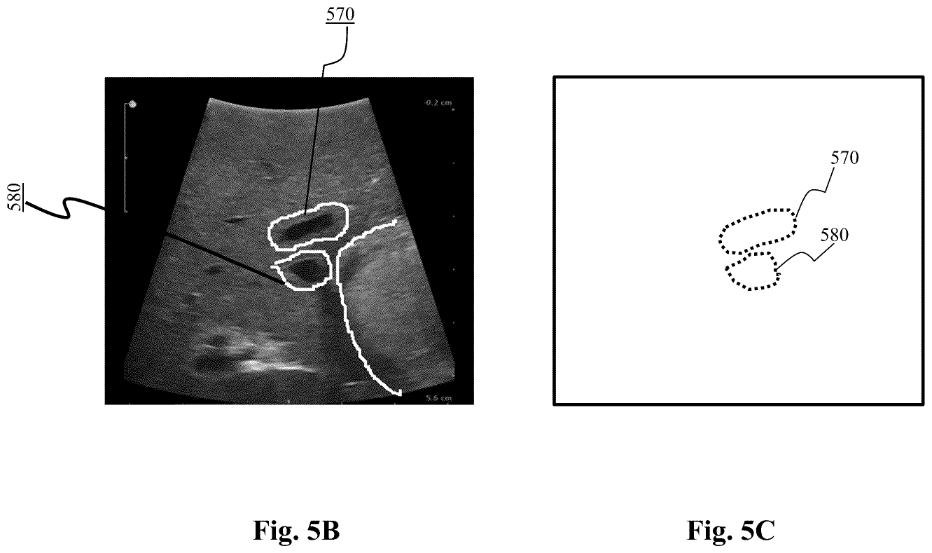

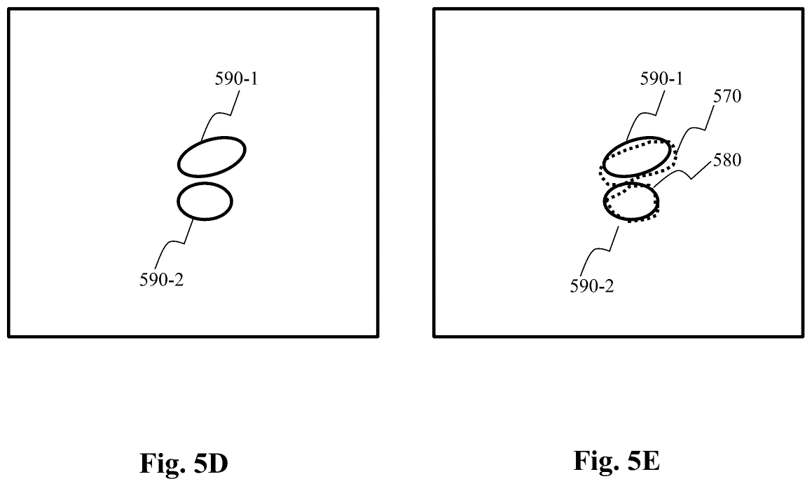

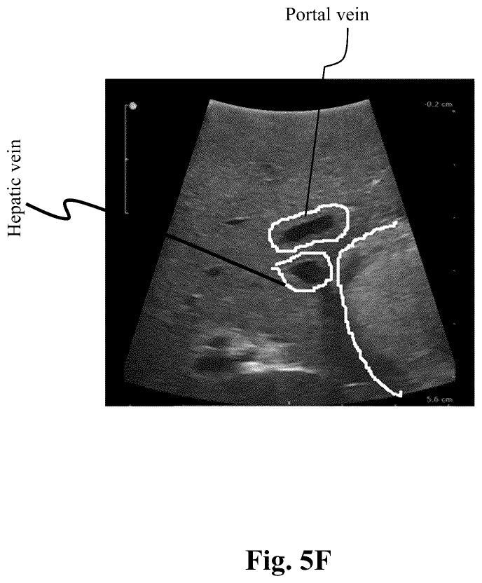

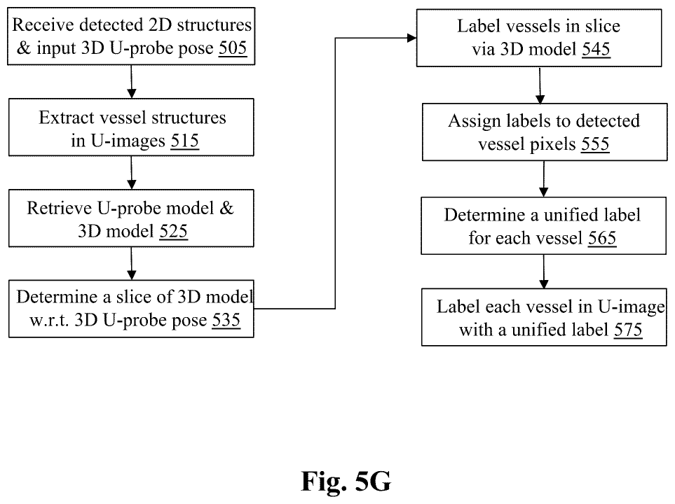

Such a 3D USPP may be utilized to achieve recognition of certain 2D structures detected from 2D ultrasound images that may correspond to only partially visible 2D anatomical structures with much noise. Based on 3D models for a target organ in the same 3D space as the estimated 3D USPP according to the present teaching, such partially visible anatomical structures may be labeled to provide effective guidance to a surgeon in a surgery. For example, based on the present teaching as disclosed herein, portal and hepatic vessel structures may be labeled as such even though they may be only partially visible in 2D ultrasound images. Based on the estimated 3D USPP of the ultrasound probe, the 3D anatomical structures (modeled in the 3D models) that should be seen by the ultrasound probe according to the known operational parameters associated with the ultrasound sensor may be determined so that the 3D structures corresponding to the visible 3D anatomical structures by the ultrasound probe with known labels may be projected onto a 2D image plane to yield 2D projected structures with ground truth labels associated therewith. Such 2D projected structures with ground truth labels may then be used to determine how the 2D structures detected from the 2D ultrasound images may be labeled. Details about using the estimated 3D USPP to label 2D structures visible from 2D ultrasound images are provided with reference to FIGS. 5A-5E.

FIG. 2A depicts an exemplary high level system diagram of a framework 200 for estimating a 3D USPP based on 2D laparoscopic images and application thereof to vessel labeling, in accordance with an embodiment of the present teaching. This illustrated framework 200 includes two parts, one for estimating the 3D USPP of an ultrasound probe in a LUS setting and the other for a specific application of the estimated 3D USPP to label vessels observed in 2D ultrasound images. This exemplary framework 200 is applied in an LUS procedure involving both a laparoscopic camera and an ultrasound probe to supply, respectively, 2D laparoscopic images and D ultrasound images. For example, the LUS procedure may correspond one for removing a tumor in a liver of a patient. In this case, 3D models 240 may be constructed for the target organ liver prior to the LUS procedure based on, e.g., various image scan data. The 3D models 240 may model different relevant parts of the target organ, including, e.g., the physical constructs of the target organ, the tumor inside the liver, and other relevant anatomical structures such as vessels.

The framework 200 according to this embodiment may take 2D images (both laparoscopic and ultrasound images) as input and label certain anatomical structures such as vessels in 2D ultrasound images to facilitate a surgeon in the LUS procedure to act according to the labeled vessels (e.g., clamping portal veins). It is understood that this exemplary application to label vessels by leveraging an estimated 3D USPP is provided merely as an illustration rather than limitation. Other applications may also be possible by using the estimated 3D USPP of an ultrasound probe in an LUS procedure to produce information or visual guidance to a user operating in the LUS procedure.

The first part of the framework 200 comprises an LP U-probe location detector 210 and a 3D U-probe pose estimator 220. The LP U-probe location detector 210 is provided for detecting the presence of a visible ultrasound probe and determining its 2D location in a 2D laparoscopic image. The 3D U-probe pose estimator 220 is provided for estimating the 3D pose, including 3D location and orientation, of the ultrasound probe based on the detected 2D location and the 3D models 240 for target organ. As discussed herein, the second part of the framework 200 is for utilizing the estimated 3D USPP to label vessel structures that are partially visible in 2D ultrasound images. To achieve that, the second part includes a U-image structure detector 250 and a 2D vessel label generator 260. The U-image structure detector 250 is provided for processing 2D ultrasound images generated based on what the ultrasound probe senses to identify 2D structures such as a region or edge points, etc. Because ultrasound images are generally a 2D partial view of a 3D structure, such detected 2D structures may not provide insufficient information to associate them with certain 3D anatomical structures, e.g., to find out whether a linear structure in 2D U-image is from a portal vein or a hepatic vein. Leveraging the estimated 3D USPP from the 3D U-probe pose estimator 220, the 2D vessel label generator 260 is provided for providing labels to 2D structures that are corresponding to vessels based on the 3D models 240.

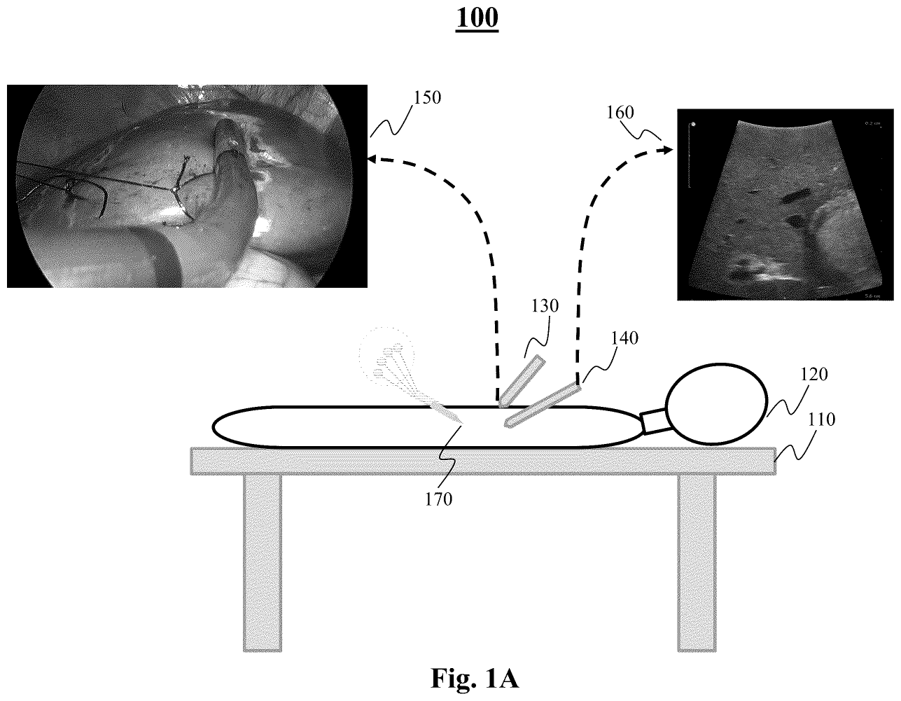

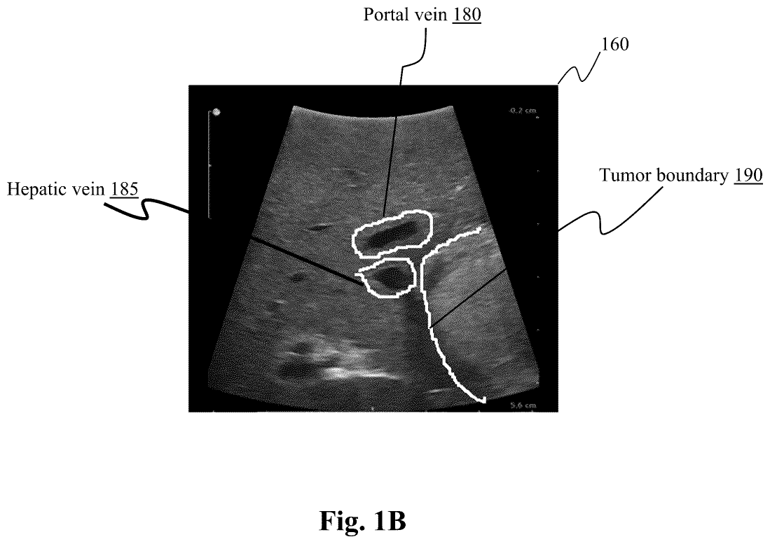

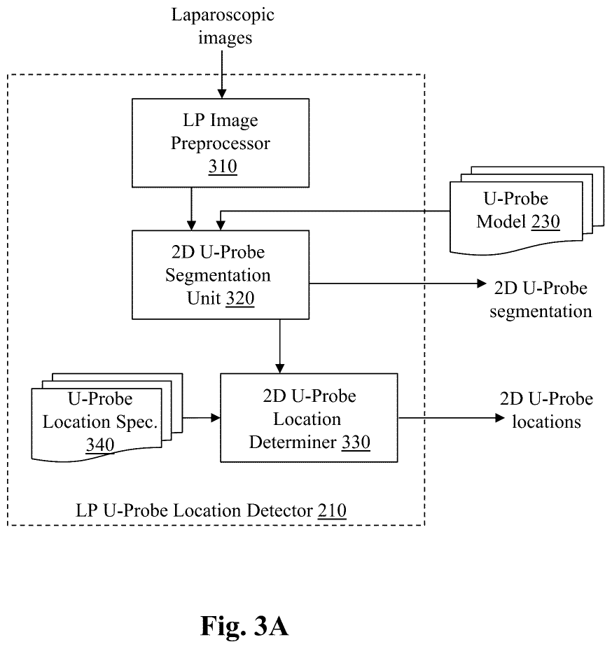

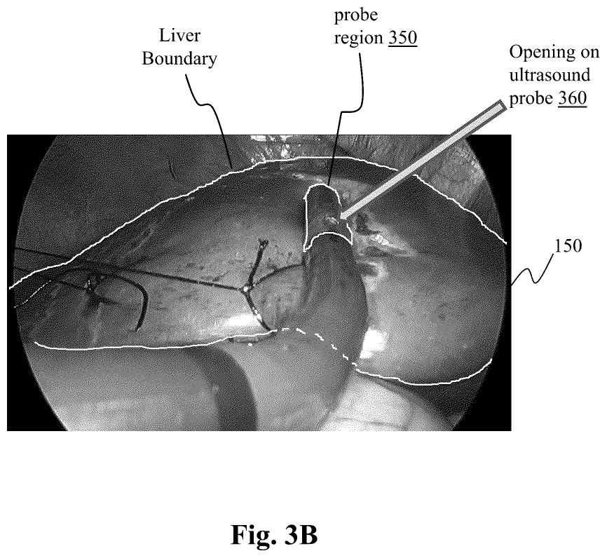

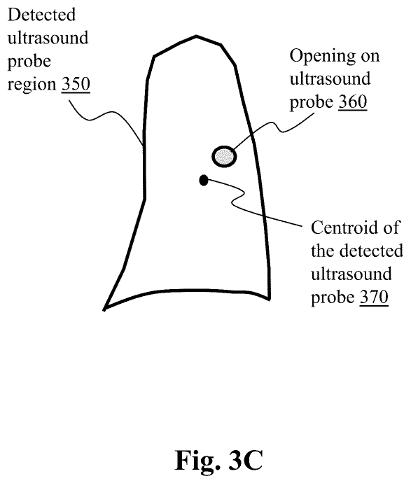

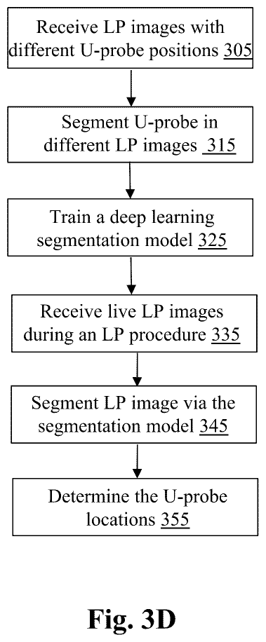

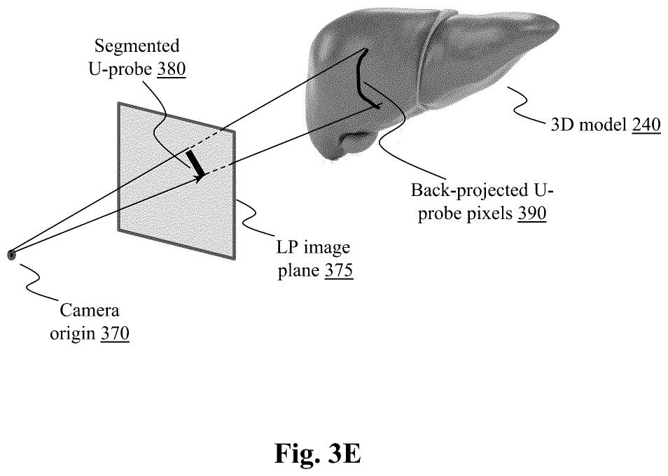

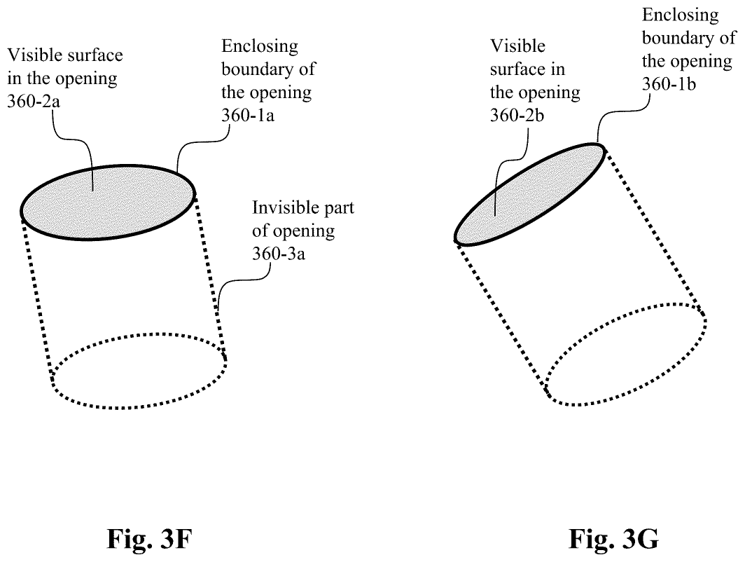

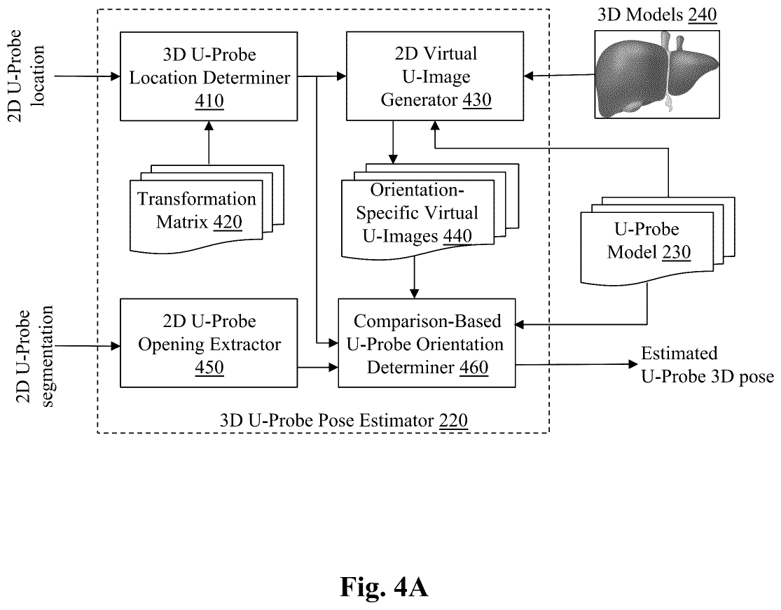

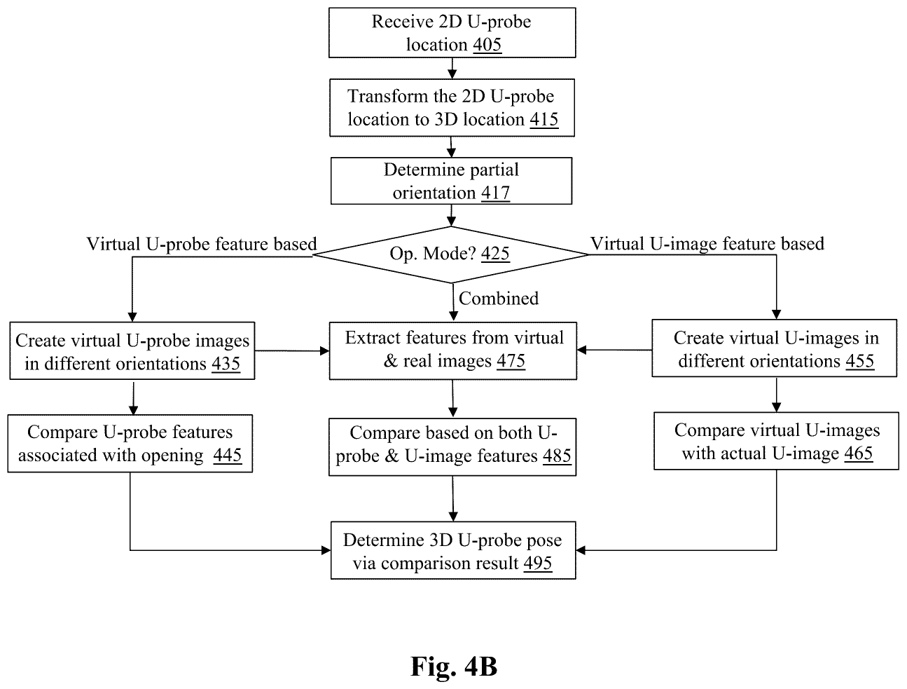

FIG. 2B is a flowchart of an exemplary process of framework 200 for estimating a 3D USPP and its application in labeling vessels, in accordance with an embodiment of the present teaching. In a LUS procedure, the LP U-probe location detector 210 receives laparoscopic images acquired by a laparoscopic camera and proceeds to detect, at 205, an area in the image corresponding to an ultrasound probe. For example, it can be seen from laparoscopic image 150 in FIG. 1A that an ultrasound probe is present therein. Based on the detected area corresponding to the ultrasound probe, the LP U-probe location detector 210 determines, at 215, the location of the U-probe in the LP image, which may be defined according to some specified definition. The 2D location of the U-probe in the laparoscopic image may then be used by the 3D U-probe pose estimator 220 to estimate, at 225, the 3D pose of the ultrasound probe detected from the 2D laparoscopic image. Such 3D pose includes a 3D coordinate (e.g., (x, y, z) in a 3D space) and orientation (i.e., pitch, roll, and yaw). Details about obtaining the 3D pose of the ultrasound probe based on a 2D location are provided with reference to FIGS. 3A-4B.

On the other hand, upon receiving the 2D ultrasound images from the ultrasound probe, the U-image structure detector 250 processes, at 235, the ultrasound images to detect, at 245, various 2D structures. Based on such detected 2D structures, the U-image structure detector 250 may estimate at 255, structures that may correspond to vessels. To label the estimated 2D vessel structures in 2D images, the 2D vessel label generator 260 may retrieve, at 265, the 3D models 240 for the target organ and may then proceed to estimate, at 275, the type or label of each vessel structure detected in the ultrasound image by leveraging the 3D USPP estimate as well as the 3D models 240. Based on the estimated label for each 2D vessel structure, the 2D vessel label generator 260 may then label, at 285, each of the 2D vessel structures in the ultrasound image.

FIG. 3A is an exemplary high level system diagram of the LP U-probe location detector 210, in accordance with an embodiment of the present teaching. As discussed above, the LP U-probe location detector 210 is provided for detecting a region in a laparoscopic image corresponding to an ultrasound probe and then determining a location thereof according to some definition. In this illustrated embodiment, the LP U-probe location detector 210 comprises an LP image preprocessor 310, a 2D U-probe segmentation unit 320, and a 2D U-probe location determiner 330. The LP image preprocessor 310 may be optionally provided for preprocessing the laparoscopic images received to generate enhanced images to facilitate further processing. In some embodiments, the input laparoscopic images may be used directly without preprocessing. The laparoscopic images, either inputted or preprocessed, may then be used by the 2D probe segmentation unit 320 to identify a region in the 2D images where the ultrasound probe is present. In some embodiments, the segmentation may be performed based on a U-probe model 230 to segment a 2D probe region in the laparoscopic image. In some embodiments, some features within this segmented region may also be extracted. In some other embodiments, exemplary images of the LP view of the U-probe and the ground truth locations of the U-probe regions may be used to train a neural network, via machine learning, for U-probe segmentation and location determination. After the training, the trained neural network may be used to segment a U-probe region and a location thereof based on any input LP image with a U-probe therein.

FIG. 3B shows an exemplary detected 2D region 350 corresponding to an ultrasound probe as it appears in a laparoscopic image, in accordance with an embodiment of the present teaching. As shown, in a laparoscopic image 150, the 2D U-probe segmentation unit 320 may detect based on, e.g., the U-probe model 230, a 2D region 350 as corresponding to an ultrasound probe. The U-probe model 230 may model the physical characteristics in terms of both the boundary and some distinction features such as an opening 360 located on the ultrasound probe. This is illustrated in FIG. 3C, which shows the exemplary segmentation result or 2D boundary 350 of an ultrasound probe detected from an 2D image and the opening 360 extracted therefrom. In some embodiments, based on a segmentation of the ultrasound probe, the 2D U-probe location determiner 330 may determine a representative 2D location of the detected probe based on, e.g., some specified configuration specified in 340 on how to determine a location based on a segmented region. In some embodiments, a representative location of a 2D region (representing the ultrasound probe boundary) may be defined as the centroid 370 of the region, as illustrated in FIG. 3C. Other ways to define the representative location of the ultrasound probe as detected may also be used.

There may be different ways to detect the location of the U-probe as it appears in a 2D image. In some embodiments, a traditional approach may be used to detect first features related to a U-probe and then classify whether the features reveal the presence of the U-probe. In some embodiments, a model may be trained via deep learning based on training data. FIG. 3D is a flowchart of an exemplary process of the LP U-probe location detector 210 to utilizing a segmentation model obtained via machine learning to identify a U-probe from a 2D image, in accordance with an embodiment of the present teaching. The location detector 210 may identify locations of a U-probe in LP images based on a model trained for segmenting a U-probe. This model may be trained offline based on training data and used to perform segmentation during an LP procedure. At 305, laparoscopic images from previous surgical procedures may be retrieved with a U-probe at various positions and orientations therein. Manual or semi-automatic segmentation may be performed to segment, at 315, the U-probes at different positions and orientations from the laparoscopic images. A deep learning neural network may be trained, at 325, with training data based on the laparoscopic images and U-probe segmentation as ground truth U-probe locations. The training may be performed offline before the surgery. During surgery, when live laparoscopic images are received at 335, they are provided to the trained model as input to the deep learning model to segment, at 345, the U-probes are they appear in the live LP images and their locations. Such determined 2D U-probe location may then be output at 355.

With the estimated 2D location of the ultrasound probe, the 3D pose of the ultrasound probe may be further estimated by the 3D U-probe pose estimator 220 (see FIG. 2A). FIG. 4A depicts an exemplary high level system diagram of the 3D U-probe pose estimator 220, in accordance with an embodiment of the present teaching. A 3D pose has 6 degrees of freedom, including (x, y, z, r, y, p), corresponding to (x, y, z) as a 3D coordinate in a 3D space as well as roll, yaw, and pitch associated with an orientation. The 3D probe pose estimator 220 is provided to estimate both the 3D coordinate and the orientation of the U-probe based on the 2D location of the U-probe estimated from 2D laparoscopic images. In this illustrated embodiment, the 3D U-probe pose estimator 220 comprises a 3D probe location determiner 410, a 2D probe opening extractor 450, a 2D virtual image generator 430, and a comparison-based probe orientation determiner 460.

The 3D probe location determiner 410 may be provided for estimating, based on a 2D probe location, a corresponding 3D location of the U-probe. The 2D U-probe opening extractor 450 may be provided for extracting, from laparoscopic images, 2D features related to the opening hole of the U-probe as it appears in 2D laparoscopic images, including but not limited to, a 2D region corresponding to the opening on the U-probe as well as its boundary points. The comparison-based probe orientation determiner 460 may be provided to estimate, based on the 2D features detected from laparoscopic images, the orientation of the U-probe in accordance with the probe model 230. In some embodiments, it is optional that the estimation of the orientation of the U-probe may also be based on 2D virtual ultrasound images 440 generated via simulation by projecting the 3D model 240 onto a 2D image plane based on the 3D coordinate estimated as well as ultrasound sensor parameters specified by the probe model 230. Such generated virtual 2D ultrasound images may be generated using different hypothesized U-probe orientations and then used to compare with the 2D ultrasound images actually acquired by the U-probe to determine the U-probe orientation.

With respect to estimating the 3D coordinate of the U-probe based on a 2D location detected from a laparoscopic image, when the laparoscopic camera is calibrated in a 3D space, each 2D pixel in a laparoscopic image may correspond to a line of sight in the 3D space which may be obtained via a transformation using a transformation matrix obtained during the calibration. When the 3D model 240 is also transformed to the same 3D coordinate system, each pixel in a laparoscopic image 375 can be back projected to the 3D model 240 in the 3D space. How to obtain such a transformation is not within the scope of the present teaching. Given that, the estimated 2D location of the U-probe in the laparoscopic image may be used to obtain, via back-projection, a 3D coordinate of the ultrasound probe. FIG. 3E depicts this exemplary scheme, where a laparoscopic camera 370 is calibrated in a 3D space in which the 3D model 240 is also displayed. With this set up, each pixel location in laparoscopic image 375 may be back projected to a 3D coordinate on the 3D model 240. Thus, the 2D U-probe segmentation 380 may be back-projected onto 390 of the 3D model 240. As such, the estimated 2D U-probe location may also be back projected to a 3D coordinate in the 3D space.

Thus, assume that such calibration is done and a transformation matrix 420 is available, the 3D U-probe location determiner 410 may take the detected 2D U-probe location and the 3D model in the camera coordinate system as inputs and then compute, using the transformation matrix, the 3D location or a 3D coordinate of the U-probe. The orientation of the U-probe (except for the rotation angle around the probe's axis) may be estimated, e.g., by fitting a 3D line to the 3D points obtained via back-projection of the 2D U-probe pixels onto the 3D model. To determine the additional degree relating to the rotation angle of the U-probe around its axis, additional image features associated with the U-probe may be detected from 2D laparoscopic images and leveraged to facilitate the estimation of the 3D orientation of the U-probe. There may be different means to estimate the 3D orientation of a U-probe given its known 3D location. One exemplary approach may be based on additional features on the U-probe as compared with what is observed from 2D laparoscopic images. Another exemplary approach is to leverage observable anatomical structures in laparoscopic images as compared with 2D virtual ultrasound images generated by projecting part of the 3D model 240 given operational parameters of the U-probe. Such 2D virtual ultrasound images may be created with respect to different orientations at the given 3D location. Yet another approach may be to integrate both the U-probe feature based and virtual 2D ultrasound image-based means to optimize the estimated 3D orientation of the U-probe.

As discussed herein, a U-probe may have an opening or a hole 360 thereon as illustrated in FIGS. 3B-3C. Features associated with the hole on a U-probe may be modeled by the U-probe model 230. FIGS. 3F-3G illustrate a modeled 3D structure of the opening 360 located on an ultrasound probe, in accordance with different embodiments of the present teaching. As seen in FIG. 3F, in some embodiments, the hole 360 may correspond to a 3D cylindrical structure embedded in a U-probe with a visible part and an invisible part. For example, the visible part of the 3D hole 360 may include an enclosing circle or boundary 360-1a of the opening and a visible portion of the interior wall area 360-2a of the cylindrical structure. The invisible part of the 3D hole 360 may include the dotted portion 360-3a. These modeled features may be utilized to estimate the orientation of a U-probe once the 3D coordinate of the U-probe is estimated.

Depending on the orientation of the U-probe, the characteristics of the visible part (e.g., the shape of the rim of the opening 360-1a and the shaped of the wall surface area 360-2a) on a U-probe may accordingly change. This is shown in FIG. 3G, where due to different orientations, the visible shapes of the rim of the opening 360-1b and accordingly of the area corresponding to the observable wall area 360-2b of the hole 360 vary from corresponding observed properties associated with 360-1a and 360-2a, respectively. Assume that the shape of the rim of the opening 360-1 is known (e.g., a circle), the shape of the rim as well as the shape of the wall area as detected from a 2D laparoscopic image (e.g., an ellipse) may then be utilized by the comparison-based U-probe orientation determiner 460 to estimate the orientation of the probe. For example, the U-probe model 230 may be used to create 2D virtual images of the U-probe with respect to different orientations and the visible 2D features of the projected U-probe may be extracted and compared with the corresponding observations obtained from the laparoscopic images. The orientation that yields the best match may be used as the estimated orientation of the U-probe.

On the other hand, with the estimated 3D location of the U-probe, given the 3D model 240 as well as the U-probe model 230, the 2D virtual U-image generator 430 may be provided to generate 2D virtual ultrasound images 440 based on slices from the 3D model 240 when viewed from different angles, where the angles are determined based on different orientations and the slices are determined based on the operational parameters of the U-probe (e.g., modeled by the U-probe model 230). The 2D image features for observed 2D structured may be detected from such 2D virtual ultrasound images and compared with that detected from the actual ultrasound images. A best match may be identified to determine the orientation that yields the best match. As discussed herein, the comparison-based U-probe orientation determiner 460 may be configured to estimate the U-probe based on any of the possible approaches according to a pre-configured operational mode.

FIG. 4B is a flowchart of an exemplary process of the 3D U-probe pose estimator, in accordance with an embodiment of the present teaching. Upon receiving the estimated 2D location of a U-probe at 405, the 3D U-probe location determiner 410 transforms, at 415, the 2D location to a 3D coordinate representing the 3D location of the U-probe based on the transformation matrix 420 and the 3D model. Partial orientation of the U-probe may be estimated, at 417, e.g., via a fitting of a linear structure to the 3D coordinates. The estimation of the remaining 3D orientation of the U-probe, i.e., rotation around the probe's axis, is performed in accordance with a configured operational mode, determined at 425. If it is configured to operate in a mode to use virtual U-probe features, the U-probe model 230 is used to create, at 435, different 2D virtual U-probe images with respect to different orientations from the estimated 3D location of the U-probe. 2D features associated with the opening of the U-probe in 2D virtual U-probe images are then compared, at 445, with that extracted from the laparoscopic images.

If it is configured to operate in a mode to use virtual ultrasound images, the 2D virtual U-image generator 430 may create, 455, 2D virtual ultrasound images with respect to different orientations of the U-probe according to both the 3D model 240 and the U-probe model 230. Such 2D virtual ultrasound images may correspond to different slices of the 3D model determined based on a given 3D pose of the U-probe (i.e., an assumed orientation as well as the estimated 3D location of the U-probe) and the operational parameters of the U-probe (i.e., the depth of signal detected by the ultrasound probe). 2D structures captured in both the virtual ultrasound images and the actual ultrasound images may be identified and compared at 465 so that the best match can be identified. An orientation corresponding to the virtual ultrasound image that gives rise to the best match may be estimated as the orientation of the U-probe.

If the operational mode is configured to estimate the orientation based on comparison results using 2D features from both virtual U-probe images and virtual ultrasound images, the virtual U-probe images and virtual ultrasound images created at 435 and 455, respectively, are used to extract, at 475, 2D features as discussed herein and are compared with, at 485, corresponding features from laparoscopic images (for U-probe) and the ultrasound images. The comparison results (from one of 445, 465, and 485 according to the operational mode) are then used to determine, at 495, the 3D orientation (e.g., based on a best match) and hence, the 3D pose of the U-probe. Such an estimated 3D U-probe pose may be applied in a LUS procedure to further assist a user such as a surgeon to receive additional guidance. As discussed herein, in some LUS procedures, a surgeon may need to clamp a portal vein nearby to stop the blood flow before cutting a tumor. In this scenario, an automated guidance as to which of the visual 2D structures seen in ultrasound images corresponds to a portal vein may be instrumental. In addition, the estimated 3D U-probe pose according to the present teaching may also be utilized to ascertain, e.g., whether 2D structures observable from ultrasound images are blood vessels or boundary of a tumor. With the 3D model 240 and the U-probe model 230 available, the 3D U-probe pose may be applied to correspond 2D structures observed in ultrasound images with 3D anatomical structures captured by the 3D model 240.

As discussed herein, an exemplary application of a 3D U-probe pose estimated according to the present teaching is to label vessels in 2D ultrasound images. One specific example application is discussed with reference to FIG. 1B, where 2D structures as they appear in 2D ultrasound images are to be labeled as different types of blood vessels, e.g., portal vein and hepatic vein. FIG. 2A illustrates the use of 3D U-probe pose in such an application, where the 2D vessel label generator 260 utilizes a 3D U-probe pose estimated in accordance with the present teaching and generates vessel labels for 2D structures detected from ultrasound images by the U-image structure detector 250 that are recognized as vessels to provide guidance to a surgeon during an LUS procedure.

Some anatomical structures including vessels may deform during a surgery. As a consequence, the shapes and the relative locations of anatomical structures may accordingly change. This poses challenges in terms of how to leverage the ground truth labels of different anatomical structures present in a virtual ultrasound image to label corresponding 2D structures detected from an actual ultrasound image. This includes the task of labeling blood vessels. The present teaching discloses an exemplary method to labeling 2D structures in ultrasound images in a LUS procedure given a known 3D ultrasound probe pose. In some embodiments, labeling 2D structures based on an estimated 3D U-probe pose may be achieved in a two-phase process according to the present teaching. In the first phase, each pixel in 2D structures detected from an actual ultrasound image may be assigned a label associated with a corresponding pixel in a virtual ultrasound image. The virtual ultrasound image may be generated based on a given 3D U-probe pose and operational parameters of the U-probe. The corresponding pixel in such a virtual ultrasound image may be selected based on some criterion, as will be discussed below. With all pixels of the detected 2D structures having assigned labels, in the second phase, a unified label for each coherent 2D structure (e.g., pixels that form roughly a circular shape) may be determined based on the labels of the pixels therein provided in the first phase based on some specified criterion. The labeling approach as discussed herein may be applied to label vessel types and details related thereto are provided with reference to FIGS. 5A-5E.

FIG. 5A depicts an exemplary high level system diagram of the 2D vessel label generator 260, in accordance with an embodiment of the present teaching. In this illustrated embodiment, the 2D vessel label generator 260 takes the 2D structures detected from ultrasound images and an estimated 3D U-probe pose as inputs and output the U-images with labels provided to 2D structures that are recognized as vessels. In this illustration, the 2D vessel label generator 260 comprises a vessel structure extractor 510, a labeled vessel projection unit 530, a vessel pixel labeling unit 540, and a vessel labeling unit 550. The vessel structure extractor 510 may be provided to extract vessel structures from the input 2D structures detected from ultrasound images and store such detected vessel structures in 520. Based on a perspective determined according to the estimated 3D U-probe pose, the labeled vessel projection unit 530 may be invoked to generate a virtual ultrasound image corresponding to a slice of the 3D model 240 at a depth determined according to the operational parameters of the U-probe specified by, e.g., the U-probe model 230. This slice may include 2D structures with ground truth labels, including the vessel type labels. For example, each of the pixels in each of the virtual vessels is assigned with a label representing, e.g., vessel type.

FIG. 5B illustrates an actual ultrasound image with some 2D structures detected therefrom, some of which may be recognized by the vessel structure extractor 510 as corresponding to vessels (e.g., 2D structures 570 and 580). The boundaries of the extracted 2D vessel structures are accordingly illustrated in FIG. 5C as dotted and enclosures. As can be seen in FIGS. 5B-5C, due to deformation or noise in ultrasound images, the 2D vessels 570 and 580 as detected may flattened or with a changed shape. FIG. 5D shows ground truth vessels 590-1 and 590-2 in the virtual ultrasound slice image generated based on the estimated 3D U-probe pose. Each of the pixels within 590-1 and 590-2 may have a ground truth label indicating, e.g., a corresponding type of vessel such as portal vein or hepatic vein. In this case, all pixels within 590-1 may be labeled as portal vein and all pixels within 590-2 may be labeled as hepatic vein. Based on the labeling, all vessel pixels connected to the labelled pixels may be assigned to the same labels. As seen, both ground truth vessels 590-1 and 590-2 have a smooth and substantially round shape.

FIG. 5E shows the ground truth vessel labels 590-1 and 590-2 superimposed on the detected 2D vessel structures 570 and 580, respectively. Although not identical in shape, size, and location, the ground truth vessels (590-1 and 59-2) and the detected vessel structures (570 and 580) do overlap, which may be leveraged to derive labels for each of the pixels in the detected 2D vessel structures. Based on the ground truth vessel labels in the virtual ultrasound slice image, the vessel pixel labeling unit 540 may be provided to carry out the first stage of the labeling, i.e., assigning a label to each pixel in each of the vessel structures 520. In some embodiments, a label assigned to each pixel in a detected vessel may be determined based on the labels of one or more corresponding pixels in the virtual ultrasound slice image. For instance, such one or more corresponding pixels in the virtual ultrasound image may be from a neighborhood defined with respect to each pixel to be labeled in a vessel detected from an ultrasound image. In some embodiments, the neighborhood may be defined as all pixels that are directly connected to the pixel to be labeled. Another definition of a neighborhood may be a neighbor of the pixel to be labeled that is closest thereto.

A corresponding neighborhood in the virtual ultrasound slice image may be accordingly identified as the one on the same pixel locations. The vessel labels associated with the pixels in this corresponding neighborhood in the virtual ultrasound image may be used to determine the vessel label to be assigned to the pixel to be labeled. For example, in some embodiments, a majority rule may be employed to determine a label to be assigned to the pixel to be labeled, e.g., a label that is associated with majority of the pixels in the corresponding neighborhood in the virtual ultrasound image. Other means to determine a label may also be deployed. Through this first stage of labeling process, all pixels within 2D vessels in 520 are assigned with a label representing a type of vessel.

Through the first stage of pixel level labeling, it is possible that pixels belonging to the same vessel may be assigned with different labels. For instance, the pixels within the illustrated vessel 570 may be assigned with different labels. As illustrated in FIG. 5E, while a substantial part of the vessel 570 overlaps with ground truth vessel 590-1 with a ground truth label, e.g., a portal vein, a small part of the 570 may overlap with 590-2 with a different label, e.g., hepatic vein. The second stage of vessel labeling may be directed to perform vessel level labeling, i.e., assigning a unified label to each of the detected vessels in 520. The vessel labeling unit 550 may be provided for that purpose. In some embodiments, identifying a unified label for each detected vessel may be accomplished based on labels assigned (in the first stage) to all pixels within the vessel. For example, the labels assigned to pixels in detected vessel 570 may form the basis to determine a unified vessel label for 570. In some embodiments, a majority rule may be employed, i.e., the label commonly assigned to majority of the pixels in a vessel may be used as the unified label for the vessel. In the example of vessel 570, although some of its pixels may be assigned label portal vein and some hepatic vein, as majority of the pixels have been labeled as portal vein, vessel 570 is assigned with a unified label as a portal vein. This is shown in FIG. 5F, where the entire 2D vessel structure is labeled as a portal vein and accordingly, another 2D vessel structure (580) is labeled as a hepatic vein.

FIG. 5G is a flowchart of an exemplary process of the 2D vessel label generator 260, in accordance with an embodiment of the present teaching. The 2D structures detected from ultrasound images and the 3D U-probe pose estimated according to the present teaching may be received, at 505, as input. To provide labels for vessels that appear in actual ultrasound images, the vessel structure extractor 510 processes the 2D structures and extracts, at 515, vessel structures 520. To facilitate the pixel labeling in the first stage, the labeled vessel projection unit 530 retrieves, at 525, the 3D model 240 and the U-probe model 230 and computes a perspective or angle of viewing with respect to the 3D model 240 based on the input 3D U-probe pose. From this viewing angle, a slice of the 3D model 240, which is perpendicular to the viewing angle, is determined, at 535, based on the operational parameters associated with the ultrasound probe as specified by the U-probe model 230. For each of the vessels visible in the slice (e.g., cross sections of the vessels), provide labels, at 545, to such pixels according to the 3D model 240. Such a created virtual ultrasound slice image with vessels labeled therein may then be leveraged to label the 2D vessel structures detected from the ultrasound images acquired during an LUS procedure.

For each of the vessel structures in 520, the vessel pixel labeling unit 540 may perform the first stage of pixel labeling by assigning, at 555, a label to each of pixels within the vessel based on the vessel pixel labels provided identified in the virtual ultrasound slice image according to the present teaching. An ultrasound image having vessel pixels assigned with pixel labels may then be processed by the vessel labeling unit 550 for the second stage for labeling each vessel detected. As discussed herein, to do so, a unified label for each vessel structure may be identified at 565, which is then assigned, at 575, to all pixels of the vessel. At this point, the ultrasound image showing partial and noisy visual information at a certain depth beneath an organ surface may have different parts marked as corresponding to different types of vessels, as illustrated in FIG. 1B. This may provide an effective guidance to a surgeon during a surgery.

Although the exemplary application of a 3D U-probe pose estimated via laparoscopic images is provided via vessel labeling, it is merely for illustration, rather than as a limitation, for the potential application of the 3D U-probe pose estimated from laparoscopic images according to exemplary embodiments of the present teaching. Other applications such as labeling other anatomical structures such as a tumor or an organ may also be realized via the illustrated labeling scheme as discussed herein.

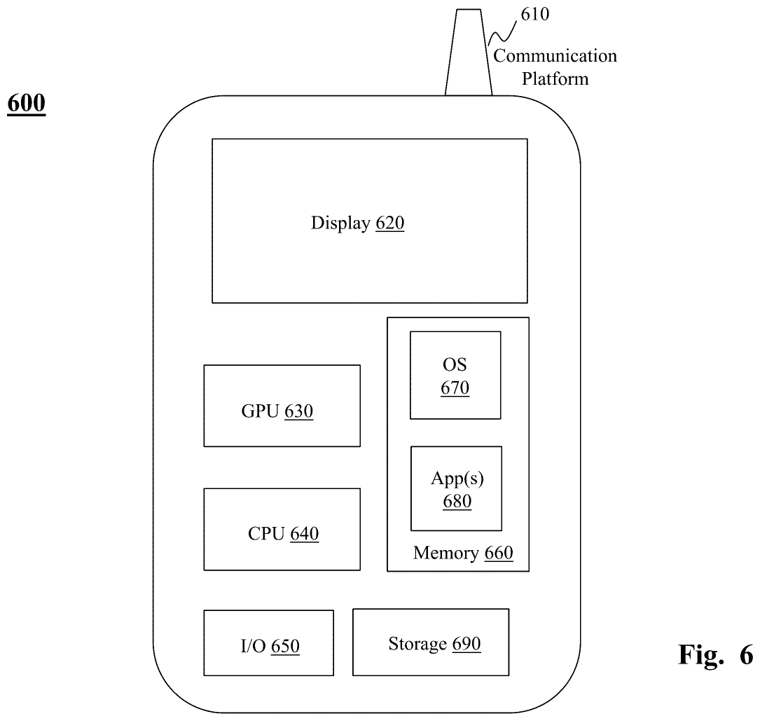

FIG. 6 is an illustrative diagram of an exemplary mobile device architecture that may be used to realize a specialized system implementing the present teaching in accordance with various embodiments. In this example, the user device on which the present teaching may be implemented corresponds to a mobile device 600, including, but not limited to, a smart phone, a tablet, a music player, a handled gaming console, a global positioning system (GPS) receiver, and a wearable computing device, or in any other form factor. Mobile device 600 may include one or more central processing units (“CPUs”) 640, one or more graphic processing units (“GPUs”) 630, a display 620, a memory 660, a communication platform 610, such as a wireless communication module, storage 690, and one or more input/output (I/O) devices 650. Any other suitable component, including but not limited to a system bus or a controller (not shown), may also be included in the mobile device 600. As shown in FIG. 6, a mobile operating system 670 (e.g., iOS, Android, Windows Phone, etc.), and one or more applications 680 may be loaded into memory 660 from storage 690 in order to be executed by the CPU 640. The applications 680 may include a user interface or any other suitable mobile apps for information analytics and management according to the present teaching on, at least partially, the mobile device 600. User interactions, if any, may be achieved via the I/O devices 650 and provided to the various components connected via network(s).

To implement various modules, units, and their functionalities described in the present disclosure, computer hardware platforms may be used as the hardware platform(s) for one or more of the elements described herein. The hardware elements, operating systems and programming languages of such computers are conventional in nature, and it is presumed that those skilled in the art are adequately familiar with to adapt those technologies to appropriate settings as described herein. A computer with user interface elements may be used to implement a personal computer (PC) or other type of workstation or terminal device, although a computer may also act as a server if appropriately programmed. It is believed that those skilled in the art are familiar with the structure, programming, and general operation of such computer equipment and as a result the drawings should be self-explanatory.

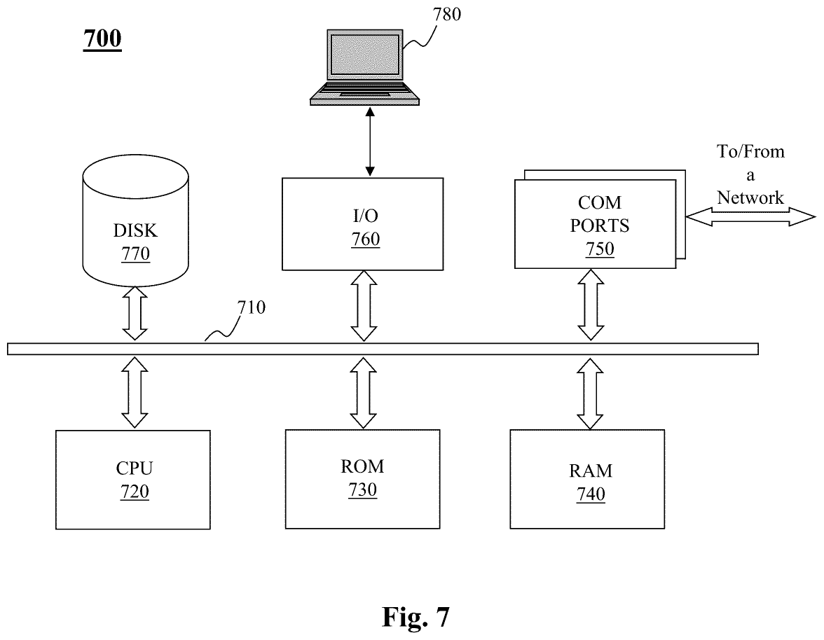

FIG. 7 is an illustrative diagram of an exemplary computing device architecture that may be used to realize a specialized system implementing the present teaching in accordance with various embodiments. Such a specialized system incorporating the present teaching has a functional block diagram illustration of a hardware platform, which includes user interface elements. The computer may be a general-purpose computer or a special purpose computer. Both can be used to implement a specialized system for the present teaching. This computer 700 may be used to implement any component or aspect of the framework as disclosed herein. For example, the information analytical and management method and system as disclosed herein may be implemented on a computer such as computer 700, via its hardware, software program, firmware, or a combination thereof. Although only one such computer is shown, for convenience, the computer functions relating to the present teaching as described herein may be implemented in a distributed fashion on a number of similar platforms, to distribute the processing load.

Computer 700, for example, includes COM ports 750 connected to and from a network connected thereto to facilitate data communications. Computer 700 also includes a central processing unit (CPU) 720, in the form of one or more processors, for executing program instructions. The exemplary computer platform includes an internal communication bus 710, program storage and data storage of different forms (e.g., disk 770, read only memory (ROM) 730, or random-access memory (RAM) 740), for various data files to be processed and/or communicated by computer 700, as well as possibly program instructions to be executed by CPU 720. Computer 700 also includes an I/O component 760, supporting input/output flows between the computer and other components therein such as user interface elements 780. Computer 700 may also receive programming and data via network communications.

Hence, aspects of the methods of information analytics and management and/or other processes, as outlined above, may be embodied in programming. Program aspects of the technology may be thought of as “products” or “articles of manufacture” typically in the form of executable code and/or associated data that is carried on or embodied in a type of machine-readable medium. Tangible non-transitory “storage” type media include any or all of the memory or other storage for the computers, processors or the like, or associated modules thereof, such as various semiconductor memories, tape drives, disk drives and the like, which may provide storage at any time for the software programming.

All or portions of the software may at times be communicated through a network such as the Internet or various other telecommunication networks. Such communications, for example, may enable loading of the software from one computer or processor into another, for example, in connection with information analytics and management. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to tangible “storage” media, terms such as computer or machine “readable medium” refer to any medium that participates in providing instructions to a processor for execution.

Hence, a machine-readable medium may take many forms, including but not limited to, a tangible storage medium, a carrier wave medium or physical transmission medium. Non-volatile storage media include, for example, optical or magnetic disks, such as any of the storage devices in any computer(s) or the like, which may be used to implement the system or any of its components as shown in the drawings. Volatile storage media include dynamic memory, such as a main memory of such a computer platform. Tangible transmission media include coaxial cables; copper wire and fiber optics, including the wires that form a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a physical processor for execution.

Those skilled in the art will recognize that the present teachings are amenable to a variety of modifications and/or enhancements. For example, although the implementation of various components described above may be embodied in a hardware device, it may also be implemented as a software only solution, e.g., an installation on an existing server. In addition, the techniques as disclosed herein may be implemented as a firmware, firmware/software combination, firmware/hardware combination, or a hardware/firmware/software combination.

While the foregoing has described what are considered to constitute the present teachings and/or other examples, it is understood that various modifications may be made thereto and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

We claim:

1. A method, comprising:
detecting a two-dimensional (2D) location of an ultrasound probe visible in a 2D laparoscopic (LP) image acquired by an LP camera previously calibrated in a three-dimensional (3D) space and inserted into a patient's body during a laparoscopic ultrasound (LPUS) procedure; and
estimating a 3D pose of the ultrasound probe deployed in the LPUS procedure based on the detected 2D location of the ultrasound probe and an ultrasound model for the ultrasound probe.

2. The method of claim 1, wherein the step of detecting a 2D location of the ultrasound image comprises:
extracting, from the 2D LP image, a 2D region corresponding to the ultrasound probe;
determining the 2D location of the ultrasound probe based on the extracted 2D region.

3. The method of claim 2, wherein
the step of extracting is based on a segmentation model for identifying an ultrasound probe obtained via machine learning based on training data; and
the 2D location of the ultrasound probe is defined as a centroid of the 2D region.

4. The method of claim 1, wherein the step of estimating a 3D pose of the ultrasound probe comprises:
transforming the 2D location into a 3D coordinate of the ultrasound probe in the 3D space based on a transformation matrix obtained in calibrating the LP camera;
estimating a 3D orientation of the ultrasound probe in accordance with the ultrasound model for the ultrasound probe; and
generating an estimated 3D pose of the ultrasound probe based on the 3D coordinate and the 3D orientation.

5. The method of claim 4, wherein the step of estimating the 3D orientation comprises:
generating multiple virtual ultrasound probe images by projecting the ultrasound model from the 3D coordinate using different 3D orientations, wherein each of the multiple virtual ultrasound probe images includes a defined feature of the ultrasound probe;
detecting a corresponding defined 2D feature of the ultrasound probe as observable in the 2D LP image;
comparing the defined feature in each of the multiple virtual ultrasound probe images with the corresponding defined 2D feature detected from 2D LP image to obtain a comparison result; and
selecting one of the 3D orientations as the estimated 3D orientation based on the comparison results.

6. The method of claim 4, wherein the step of estimating the 3D orientation comprises:
generating multiple virtual ultrasound images based on slices of a 3D model, which are obtained based on the estimated 3D coordinate, different 3D orientations of the ultrasound probe, and the ultrasound model;
comparing each of the multiple virtual ultrasound images with a 2D ultrasound image acquired by the ultrasound probe to obtain a comparison result; and
selecting one of the different possible 3D orientations as the estimated 3D orientation based on the comparison results.

7. The method of claim 1, further comprising labeling, based on the estimated 3D pose of the ultrasound probe, at least one blood vessel in a 2D ultrasound image acquired by the ultrasound probe.

8. The method of claim 7, wherein the step of labeling the at least one blood vessel comprises:
detecting 2D structures in the 2D ultrasound image;
accessing a 3D model for a target, representing a target organ and related anatomical structures including blood vessels;
determining a slice of the 3D model based on the 3D pose of the ultrasound probe and the ultrasound model;
generating a virtual 2D blood vessel image based on each blood vessel present in the 2D slice; and
providing labels to the 2D structures that correspond to blood vessels.

9. A machine readable and non-transitory medium having information recorded thereon, wherein the information, when ready by the machine, causes the machine to perform the following steps:
detecting a two-dimensional (2D) location of an ultrasound probe visible in a 2D laparoscopic (LP) image acquired by an LP camera previously calibrated in a three-dimensional (3D) space and inserted into a patient's body during a laparoscopic ultrasound (LPUS) procedure; and
estimating a 3D pose of the ultrasound probe deployed in the LPUS procedure based on the detected 2D location of the ultrasound probe and an ultrasound model for the ultrasound probe.

10. The medium of claim 9, wherein the step of detecting a 2D location of the ultrasound image comprises:
extracting, from the 2D LP image, a 2D region corresponding to the ultrasound probe;
determining the 2D location of the ultrasound probe based on the extracted 2D region.

11. The medium of claim 10, wherein
the step of extracting is based on a segmentation model for identifying an ultrasound probe obtained via machine learning based on training data; and
the 2D location of the ultrasound probe is defined as a centroid of the 2D region.

12. The medium of claim 9, wherein the step of estimating a 3D pose of the ultrasound probe comprises:
transforming the 2D location into a 3D coordinate of the ultrasound probe in the 3D space based on a transformation matrix obtained in calibrating the LP camera;
estimating a 3D orientation of the ultrasound probe in accordance with the ultrasound model for the ultrasound probe; and
generating an estimated 3D pose of the ultrasound probe based on the 3D coordinate and the 3D orientation.

13. The medium of claim 12, wherein the step of estimating the 3D orientation comprises:
generating multiple virtual ultrasound probe images by projecting the ultrasound model from the 3D coordinate using different 3D orientations, wherein each of the multiple virtual ultrasound probe images includes a defined feature of the ultrasound probe;
detecting a corresponding defined 2D feature of the ultrasound probe as observable in the 2D LP image;
comparing the defined feature in each of the multiple virtual ultrasound probe images with the corresponding defined 2D feature detected from 2D LP image to obtain a comparison result; and
selecting one of the 3D orientations as the estimated 3D orientation based on the comparison results.

14. The medium of claim 12, wherein the step of estimating the 3D orientation comprises:
generating multiple virtual ultrasound images based on slices of a 3D model, which are obtained based on the estimated 3D coordinate, different 3D orientations of the ultrasound probe, and the ultrasound model;
comparing each of the multiple virtual ultrasound images with a 2D ultrasound image acquired by the ultrasound probe to obtain a comparison result; and
selecting one of the different possible 3D orientations as the estimated 3D orientation based on the comparison results.

15. The medium of claim 9, wherein, the information, when read by the machine, further causes the machine to perform the step of labeling, based on the estimated 3D pose of the ultrasound probe, at least one blood vessel in a 2D ultrasound image acquired by the ultrasound probe.

16. The medium of claim 15, wherein the step of labeling the at least one blood vessel comprises:
detecting 2D structures in the 2D ultrasound image;
accessing a 3D model for a target, representing a target organ and related anatomical structures including blood vessels;
determining a slice of the 3D model based on the 3D pose of the ultrasound probe and the ultrasound model;
generating a virtual 2D blood vessel image based on each blood vessel present in the 2D slice; and
providing labels to the 2D structures that correspond to blood vessels.

17. A system, comprising:
an LP U-probe location detector implemented by a processor and configured for detecting a two-dimensional (2D) location of an ultrasound probe visible in a 2D laparoscopic (LP) image acquired by an LP camera previously calibrated in a three-dimensional (3D) space and inserted into a patient's body during a laparoscopic ultrasound (LPUS) procedure; and
a 3D U-probe pose estimator implemented by a processor and configured for estimating a 3D pose of the ultrasound probe deployed in the LPUS procedure based on the detected 2D location of the ultrasound probe and an ultrasound model for the ultrasound probe.

18. The system of claim 17, wherein the step of detecting a 2D location of the ultrasound image comprises:
extracting, from the 2D LP image, a 2D region corresponding to the ultrasound probe;
determining the 2D location of the ultrasound probe based on the extracted 2D region.

19. The system of claim 18, wherein
the step of extracting is based on a segmentation model for identifying an ultrasound probe obtained via machine learning based on training data; and
the 2D location of the ultrasound probe is defined as a centroid of the 2D region.

20. The system of claim 17, wherein the step of estimating a 3D pose of the ultrasound probe comprises:
transforming the 2D location into a 3D coordinate of the ultrasound probe in the 3D space based on a transformation matrix obtained in calibrating the LP camera;
estimating a 3D orientation of the ultrasound probe in accordance with the ultrasound model for the ultrasound probe; and
generating an estimated 3D pose of the ultrasound probe based on the 3D coordinate and the 3D orientation.

21. The system of claim 20, wherein the step of estimating the 3D orientation comprises:
generating multiple virtual ultrasound probe images by projecting the ultrasound model from the 3D coordinate using different 3D orientations, wherein each of the multiple virtual ultrasound probe images includes a defined feature of the ultrasound probe;
detecting a corresponding defined 2D feature of the ultrasound probe as observable in the 2D LP image;
comparing the defined feature in each of the multiple virtual ultrasound probe images with the corresponding defined 2D feature detected from 2D LP image to obtain a comparison result; and
selecting one of the 3D orientations as the estimated 3D orientation based on the comparison results.

22. The system of claim 20, wherein the step of estimating the 3D orientation comprises:
generating multiple virtual ultrasound images based on slices of a 3D model, which are obtained based on the estimated 3D coordinate, different 3D orientations of the ultrasound probe, and the ultrasound model;
comparing each of the multiple virtual ultrasound images with a 2D ultrasound image acquired by the ultrasound probe to obtain a comparison result; and
selecting one of the different possible 3D orientations as the estimated 3D orientation based on the comparison results.

23. The system of claim 17, further comprising a 2D vessel label generator implemented by a processor and configured for labeling, based on the estimated 3D pose of the ultrasound probe, at least one blood vessel in a 2D ultrasound image acquired by the ultrasound probe.

24. The system of claim 23, wherein the step of labeling the at least one blood vessel comprises:
detecting 2D structures in the 2D ultrasound image;
accessing a 3D model for a target, representing a target organ and related anatomical structures including blood vessels;
determining a slice of the 3D model based on the 3D pose of the ultrasound probe and the ultrasound model;
generating a virtual 2D blood vessel image based on each blood vessel present in the 2D slice; and
providing labels to the 2D structures that correspond to blood vessels.
